SEQUENCE LISTING (49 sequences)

SEQ ID NO: 1 - DNA, 17 bp, Artificial Sequence
  Olligonucleotide CF4
aatagggctcgagcggc

SEQ ID NO: 2 - DNA, 27 bp, Artificial Sequence
  Oligonucleotide CF5
ggatcctaatacgactcactatagggc

SEQ ID NO: 3 - DNA, 26 bp, Artificial Sequence
  Oligonucleotide CF35
cgattcgacatagaaataaattggag

SEQ ID NO: 4 - DNA, 22 bp, Artificial Sequence
  Oligonucleotide CF43
gtttggtgacctatagtcagtg

SEQ ID NO: 5 - DNA, 22 bp, Artificial Sequence
  Oligonucleotide CF45
tggatggcatgaatgtatagat

SEQ ID NO: 6 - DNA, 28 bp, Artificial Sequence
  Oligonucleotide TB59
aaagaagcataacatatgtcaaaacaag

SEQ ID NO: 7 - DNA, 26 bp, Artificial Sequence
  Oligonucleotide TB85
acacttcatcactttgataccccaga

SEQ ID NO: 8 - DNA, 27 bp, Artificial Sequence
  Oligonucleotide TB86
ccatttgaccatgagaagacatccatc

SEQ ID NO: 9 - DNA, 31 bp, Artificial Sequence
  Oligonucleotide TB95
aaatctccatttgaatgaagtgcctctgggg

SEQ ID NO: 10 - DNA, 33 bp, Artificial Sequence
  Oligonucleotide TB96
gtccacaaagtgctcaatattatcccgattgag

SEQ ID NO: 11 - DNA, 31 bp, Artificial Sequence
  Oligonucleotide TB100
agggcgtcagagaatctccaacccatatacc

SEQ ID NO: 12 - DNA, 36 bp, Artificial Sequence
  Oligonucleotide TB103
ggaattccatatgcggataataaatatatataaacg

SEQ ID NO: 13 - DNA, 37 bp, Artificial Sequence
  Oligonucleotide TB104
ggaattccatatgcgattcacaaaaaataggcacacg

SEQ ID NO: 14 - DNA, 23 bp, Artificial Sequence
  Oligonucleotide TB107
gcaggagtggacgaagaagctcc

SEQ ID NO: 15 - DNA, 31 bp, Artificial Sequence
  Oligonucleotide TB117
ggatcccatatgtaaggagcataaaaatggc

SEQ ID NO: 16 - DNA, 44 bp, Artificial Sequence
  Oligonucleotide CF4
ctaatacgactcactatagggctcgagcggccgcccgggcaggt

SEQ ID NO: 17 - DNA, 30 bp, Artificial Sequence
  Oligonucleotide CF8
tctagaggtaccttctcgtgctttgtccgg

SEQ ID NO: 18 - DNA, 28 bp, Artificial Sequence
  Oligonucleotide CF9
taccgtccccctaggaaacctcttgcac

SEQ ID NO: 19 - DNA, 31 bp, Artificial Sequence
  Oligonucleotide CF10
tgacttactggggatcaagcctgattgggag

SEQ ID NO: 20 - DNA, 31 bp, Artificial Sequence
  Oligonucleotide CF11
aagtacatccgcaactgtccatactctgatg

SEQ ID NO: 21 - DNA, 25 bp, Artificial Sequence
  Oligonucleotide CF14
gtttttcgtgtgcctattttttgtg

SEQ ID NO: 22 - DNA, 22 bp, Artificial Sequence
  Oligonucleotide CF15
gagcgcatcgaaaatgctgttg

SEQ ID NO: 23 - DNA, 21 bp, Artificial Sequence
  Oligonucleotide CF16
ctcagtgtaaagaggaaatcc

SEQ ID NO: 24 - DNA, 22 bp, Artificial Sequence
  Oligonucleotide CF17
gagtttcaatggtcttgtctgg

SEQ ID NO: 25 - DNA, 25 bp, Artificial Sequence
  Oligonucleotide CF18
cttgaaaagcctgagggctggttac

SEQ ID NO: 26 - DNA, 22 bp, Artificial Sequence
  Oligonucleotide CF19
cttgacctttggtacctttgac

SEQ ID NO: 27 - DNA, 22 bp, Artificial Sequence
  Oligonucleotide CF20
gatagtcacacggctactcacg

SEQ ID NO: 28 - DNA, 22 bp, Artificial Sequence
  Oligonucleotide CF21
cgtgagtagccgtgtgactatc

SEQ ID NO: 29 - DNA, 22 bp, Artificial Sequence
  Oligonucleotide CF22
gtccatagagtttggatccaag

SEQ ID NO: 30 - DNA, 22 bp, Artificial Sequence
  Oligonucleotide CF23
gtcaaaggtaccaaaggtcaag

SEQ ID NO: 31 - DNA, 21 bp, Artificial Sequence
  Oligonucleotide CF24
ccagaaattcgcgatatgaac

SEQ ID NO: 32 - DNA, 21 bp, Artificial Sequence
  Oligonucleotide CF25
gaatgaatccagataaggtgc

SEQ ID NO: 33 - DNA, 24 bp, Artificial Sequence
  Oligonucleotide CF26
gatatcttcaactcatgggattac

SEQ ID NO: 34 - DNA, 22 bp, Artificial Sequence
  Oligonucleotide CF27
caagattctcaccagttttatg

SEQ ID NO: 35 - DNA, 21 bp, Artificial Sequence
  Oligonucleotide CF28
gctgcgatgcttatgattacc

SEQ ID NO: 36 - DNA, 20 bp, Artificial Sequence
  Oligonucleotide CF29
gctaccaatgctgacaatag

SEQ ID NO: 37 - DNA, 21 bp, Artificial Sequence
  Oligonucleotide CF31
cctaagcagtttctcaagttg

SEQ ID NO: 38 - DNA, 20 bp, Artificial Sequence
  Oligonucleotide CF32
catgttgcctatcgtccagc

SEQ ID NO: 39 - DNA, 26 bp, Artificial Sequence
  Oligonucleotide CF36
ctatagtcagtgtggtttagacaagc

SEQ ID NO: 40 - DNA, 22 bp, Artificial Sequence
  Oligonucleotide CF39
gattatgctgaatcaaatagtc

SEQ ID NO: 41 - DNA, 22 bp, Artificial Sequence
  Oligonucleotide CF40
gagcacgatagtagtcaatcac

SEQ ID NO: 42 - DNA, 23 bp, Artificial Sequence
  Oligonucleotide CF41
caatttttgactgatacgatggc

SEQ ID NO: 43 - DNA, 22 bp, Artificial Sequence
  Oligonucleotide CF42
ctgttcttccaactttttcagc

SEQ ID NO: 44 - DNA, 22 bp, Artificial Sequence
  Oligonucleotide CF44
atctatacattcatgccatcca

SEQ ID NO: 45 - DNA, 18 bp, Artificial Sequence
  Nucleotide
agtttaaaatctttattc

SEQ ID NO: 46 - DNA, 18 bp, Artificial Sequence
  PCR walk 6-9 of the GP1223 insert Sequence
ttgacaaaattgtataat

SEQ ID NO: 47 - DNA, 29 bp, Artificial Sequence
  PCR walk 6-9 of the GP1223 insert Sequence
ttgacagatgtaatatctggtgttacaat

SEQ ID NO: 48 - DNA, 220 bp, Artificial Sequence
  Dyad symmetry sequence followed by a stretch of thymidine residue
ctaagataaaaaagaagctcagtgcgagagggggatttcctctttacactgagttttttgtttggaatttttagtttaaaatctttattcagtttaaaatctttattcagtttaaaatctttattcagtttaaaatctttattcagtttaaaattatttcgaaatagaataaaattcttgacagatgtaatatctggtgttacaataattaaaaatcgat

SEQ ID NO: 49 - DNA, 220 bp, Artificial Sequence
  Dyad symmetry sequence followed by a stretch of thymidine residue
gattctattttttcttcgagtcacgctctccccctaaaggagaaatgtgactcaaaaaacaaaccttaaaaatcaaattttagaaataagtcaaattttagaaataagtcaaattttagaaataagtcaaattttagaaataagtcaaattttaataaagctttatcttattttaagaactgtctacattatagaccacaatgttattaatttttagcta

(19) United States
(12) Patent Application Publication
(10) Pub. No.: US 2005/0042756 A1
(43) Pub. Date: Feb. 24, 2005

(54) Intergenic and intragenic integration sites for foreign gene expression in recombinant S. gordonii strains

(76) Inventors: Christine A. Franke, Albany, OR (US); Tove C. Bolken, Jefferson, OR (US); Kevin F. Jones, Albany, OR (US); Dennis E. Hruby, Albany, OR (US)

Correspondence Address: BURNS DOANE SWECKER & MATHIS L L P, POST OFFICE BOX 1404, ALEXANDRIA, VA, 22313-1404, US

(21) Appl. No.: 10/698,439

(22) Filed: Nov. 3, 2003

Related U.S. Application Data
(63) Continuation-in-part of application No. PCT/US01/05493, filed on Feb. 22, 2001.
(60) Provisional application No. 60/184,645, filed on Feb. 24, 2000.

(51) Int. Cl. C12N 15/74; C12N 1/21

(52) U.S. Cl. 435/471; 435/252.3

(57) ABSTRACT

The present invention provides two new chromosomal integration sites for expression of foreign genes have been developed in Streptococcus gordonii (S. gordonii). One integration site is intergenic between orfA and orfB in an operon of unknown function. The other site is intragenic within the lacG gene, which encodes phospho-β-galactosidase, and is part of the lactose (lac) operon. The emm6 gene from Streptococcus pyogenes was integrated in a stable configuration into the chromosome of S. gordonii at each of these integration sites, and in both cases the recombinant bacteria expressed the M6 protein on their surface. Furthermore, expression from the lacG site within the lactose operon was shown to be regulated by growth on lactose. Identification of these new chromosomal insertion sites provides the ability to express multiple foreign genes from the same recombinant and the potential for modulating expression in vitro or in vivo by the use of a biosynthetic metabolite.

### CONTINUING APPLICATION DATA

This application is a continuation-in-part of PCT/US01/05493, filed Feb. 22, 2001, which claims priority benefit of U.S. provisional application 60/184,645, filed Feb. 24, 2000. The contents of these prior applications are hereby incorporated by reference herein.

### U.S. GOVERNMENT SUPPORT

This work was supported in part by a grant from NIH (AI46176-01A1).

### BACKGROUND OF THE INVENTION

1. Field of the Invention

The present invention provides vectors for insertion of a heterologous DNA molecule into the genome of a gram-positive bacterium, such as the gram-positive commensal bacteria Streptococcus gordonii. Bacteria transformed with the vectors of the present invention will express the heterologous DNA, and can be used to produce the protein encoded by that DNA in vitro or in vivo.

2. Description of the Related Art

Streptococcus gordonii (S. gordonii) is a commensal bacteria of the human oral cavity. Recently, there has been a great deal of interest in engineering S. gordonii for use as a vaccine delivery vector. To that end, a large number of heterologous antigens have been expressed on the surface of S. gordonii (7, 9, 13) and these live recombinant bacteria have been shown to colonize the oral mucosa of recipient animals, inducing both a local and a systemic immune response (7).

Pozzi and coworkers made the initial S. gordonii chromosomal recombinants by randomly inserting the chloramphenicol transferase (cat) gene into the chromosome (11) and selecting the recombinant that showed the highest level of CAT activity. This recombinant then became the recipient parental strain and heterologous genes were inserted into the bacterial chromosome replacing the cat gene. Unfortunately, this method inserted genes into an unknown locus and rearranged the chromosome of wild type S. gordonii (3).

The following publications are representative of the state of the art
1. Bollet, C., et al. (1991) A simple method for the isolation of chromosomal DNA from Gram positive or acid-fast bacteria. Nucl. Acids Res. 19:1955.
2. Fischetti, V. A., et al. (1985) Size variation of the M protein in group A streptococci. J. Exp. Med. 161:1384-1401.
3. Franke, C. A., et al. (2001) Studies on the genomic organization of recombinant Streptococcus gordonii and development of a novel intergenic integration site for foreign gene expression. J. Mol. Microbiol. Biotechnol. 3: 545-555.
4. Jones, K. F., et al.(1986) Immunochemical localization and amino acid sequences of cross reactive epitopes within a streptococcal M6 protein. J. Exp. Med. 164:1226-1238.
5. Jones, K. F., et al. (1988). Spontaneous M6 protein size mutants of group A streptococci display variation in antigenic and opsonogenic epitopes. Proc. Natl. Acad. Sci. USA. 85:8271-8275.
6. Maniatis, T., et al. (1982). Molecular cloning: a laboratory manual. Cold Spring Harbor Laboratory, Cold Spring Harbor, N.Y.
7. Medaglini, D., et al. (1995). Mucosal and systemic immune responses to a recombinant protein expressed on the surface of the oral commensal bacterium Streptococcus gordonii after oral colonization. Proc. Natl. Acad. Sci. 92:6868-6872.
8. Payne, J., et al. (1996). Exploitation of a chromosomally integrated lactose operon for controlled gene expression in Lactococcus lactis. FEMS Microbiol. Lett. 136:19-24.
9. Pozzi, G., et al. (1992) Delivery and expression of heterologous antigen on the surface of streptococci. Infect. Immun. 60:1902-1907.
10. Pozzi, G., et al. (1990) Method and parameters for genetic transformation of Streptococcus sanguis Challis. Res. Microbiol. 141:659-670.
11. Pozzi, G., et al. (1988) Host-vector system for integration of recombinant DNA into chromosomes of transformable and non-transformable streptococci. J. Bact. 170:1969-1972.
12. Pozzi, G. and M. R. Oggioni. 1996. A host-vector system for heterologous gene expression in Streptococcus gordonii. Gene. 169:85-90.
13. Pozzi, G., et al. (1992). Expression of M6 protein gene of Streptococcus pyogenes in Streptococcus gordonii after chromosomal integration and transcriptional fusion. Res. Microbiol. 143:449-457.
14. Roe, B. A., et al. Streptococcal Genome Sequencing Project funded by USPHS/NIH grant #AI38406
15. Rosey, E. L. and G. C. Stewart. 1992. Nucleotide and deduced amino acid sequences of the lacR, lacABCD, and lacEF genes encoding the repressor, tagatose 6-phosphate genecluster, and sugar-specific PTS components of the lactose operon of Streptococcus mutans. J. Bact. 174:6159-6170.
16. Shiroza, T. and H. K. Kuramitsu. (1993). Construction of a model secretion system for oral streptococci. Infect. Immun. 61:3745-3755.
17. Siebert, P. D., et al. (1995). An improved PCR method for walking in uncloned genomic DNA. Nucleic Acids Research. 23: 1087-1088.
18. Simons, G., et al. (1993). Integration and Gene Replacement in the Lactococcus lactis lac Operon: Induction of a cryptic phospho-β-glucosidase in LacG-deficient strains. J. Bact. 175:5168-5175.
19. Sinha, R. P.1991. Genetic characterization of partial lactose-fermenting revertants from lactose-negative mutants of lactococci. Can. J. Microbiol. 37:281-286.
20. Van Rooijen, R. J., et al. (1992). Characterization of the Lactococcus lactis lactose operon promoter: contribution of flanking sequences and LacR repressor to promoter activity. J. Bact. 174:2273-2280.
21. Bolken, T. C. et al. (2001). Identification of an intragenic integration site for foreign gene expression in recombinant Streptococcus gordonii strains. App. Microbiol. Biotech. Appl. Microbiol. Biotechnol. 55:192-197.

With the current interest in using commensal Gram-positive bacteria as vaccine delivery vectors, there is a need for additional clean, stable insertion sites that do not appreciably disrupt the bacterial chromosome. Furthermore, having multiple expression sites makes it possible to create a vaccine for more than one antigen, and/or to co-express an adjuvant with the antigen.

### SUMMARY OF THE INVENTION

Briefly, the present invention provides stable insertion sites at distinct loci within the S. gordonii chromosome without genetically rearranging it or causing significant changes in the growth characteristics of the recombinant bacteria. Two such insertion sites were established. One is intergenic between two unknown open reading frames, orfA and orfB, downstream of the promoter that Pozzi et al. has previously used for protein expression (11). The second site is intragenic within the lacG gene, which is part of the lac operon. This second site is inducible by growth in media containing lactose.

With the foregoing and other objects, advantages and features of the invention that will become hereinafter apparent, the nature of the invention may be more clearly understood by reference to the following detailed description of the preferred embodiments of the invention and to the appended claims.

### BRIEF DESCRIPTION OF THE DRAWINGS

FIG. 1. Integration of the M/aphIII cassette into the chromosome of S. gordonii. A Ndel site was introduced between orf A and orf B in pCR2.1:635 yielding p635(Ndel). The M/aphIII cassette has been inserted at the Ndel site between orf A and orf B in p635 (Ndel) yielding the recombinant plasmid p635:M/aphIII. V288 is the recipient S. gordonii strain. OrfA and orf B provided homologous sequence for recombination of p635:M/aphIII into the chromosome of V288 resulting in strain SP-02. Primers CF43 and CF45 are located as shown.

FIG. 2. (A) Partial sequence from the lac operon in S. gordonii. A walk upstream in the chromosome of V288 from the initial TB85-TB86 PCR product produced the 1733 bp CF6-TB86 product shown and this was cloned into pCR2.1 producing pCR2.1:6-86. Walking primers TB95, TB96 and TB100 are located as shown. pLacG was made by introducing a Ndel site at amino acids 94 and 95 in the lacG gene.

(B) The M/aphIII cassette was inserted at the Ndel site producing pLacG:M/aphIII. The predicted lac operon in S. gordonii is shown. The homologous lacE/G flanks around the M/aphIII cassette allowed for recombination into the chromosome resulting in strain SP-04. Primers TB107 and TB96 are located as shown.

FIG. 3. Southern blot analysis depicting insertion of the M/aphIII cassette into 6-35 site.

(A) Schematic representation of the wild type V288 and mutant SP-02 chromosomes. The 6-36 and M/aphIII DNA probes are shown with dashed lines. The genomic DNA was digested with Clal and Smal.

(B) Southern blot of genomic DNA from V288 (lane 1) and SP-02 (lane 2) probed with 6-35 probe.

(C) Southern blot of M/aphIII DNA fragment (lane 3), V288 genomic DNA (lane 4) and SP-02 genomic DNA (lane 5) probed with M/aphIII probe.

FIG. 4. Southern blot analysis depicting chromosomal insertion of the M/aphIII cassette into the lacG orf.

(A) Schematic representation of wild type V288 and mutant SP-04 chromosomes. The lacG and M/aphIII DNA probes are shown with dashed lines. The genomic DNA was digested with Smal and Xbal.

(B) Southern blot of genomic DNA from V288 (lane 1) and SP-04 (lane 2) probed with lacG probe.

(C) Southern blot of M/aphIII DNA fragment (lane 3), V288 genomic DNA (lane 4) and SP-04 genomic DNA (lane 5) probed with M/aphIII probe.

FIG. 5. Competition ELISA with M protein surface expressing strains versus coli M6 protein. Each graph shows percent inhibition of binding of mAB 10F5 to coli M6 protein by decreasing concentrations of cells.

(A) Strains are shown as in the legend.

(B) Strains were grown in M17 broth supplemented with lactose (M17L) or glucose (M17G).

FIG. 6. Results of chromosomal walks upstream and downstream of the GP1223 insert.

FIG. 7. A: The alignment of the gram-positive promoter consensus with the sequence determined from “PCR walk 6-9” of the GP1223 insert.

B. A sequence containing dyad symmetry followed by a stretch of thymidine residues, approximately 150 nucleotides upstream of the −35 region, that conforms to a prokaryotic factor-independent RNA polymerase terminator sequence.

FIG. 8. A: proposed structure of the 3057-bp Clal fragment present as a single copy in GP204 and duplicated on either side of the M6 insertion site of GP1223.

B. Corroboration of this proposed genomic structure as demonstrated by Southern blot analyses.

FIG. 9. A: Schematic representation of the transcription units predicted from the parental (GP204)) and recombinant (GP1223) S. gordonii strains. Location of promoters (P1, P2) and terminators (T1, T2, T3) are indicated relative to gene order. Predicted transcripts and sizes are indicated as dashed arrows above the maps. Probes (*1, *2) utilized in Northern analyses are localized by solid bars below the maps.

B: Northern blot analysis of total RNA purified from S. gordonii strains GP204 and GP1223. Probes utilized are indicated above the blots and the size (nt) of transcripts detected are indicated to the right of the blots.

### DETAILED DESCRIPTION OF THE INVENTION

The present invention provides two new chromosomal integration sites for expression of foreign genes in gram positive bacteria, such as Streptococcus gordonii (S. gordonii). One integration site is intergenic between orfA and orfB in an operon of unknown function. The other site is intragenic within the lacG gene, which encodes phospho-β-galactosidase, and is part of the lactose (lac) operon. The emm6 gene from Streptococcus pyogenes was integrated in a stable configuration into the chromosome of S. gordonii at each of these integration sites, and in both cases the recombinant bacteria expressed the M6 protein on their surface.

Furthermore, expression from the lacG site within the lactose operon was shown to be regulated by growth on lactose. Identification of these new chromosomal insertion sites provides the ability to express multiple foreign genes from the same recombinant and the potential for modulating expression in vitro or in vivo by the use of a biosynthetic metabolite.

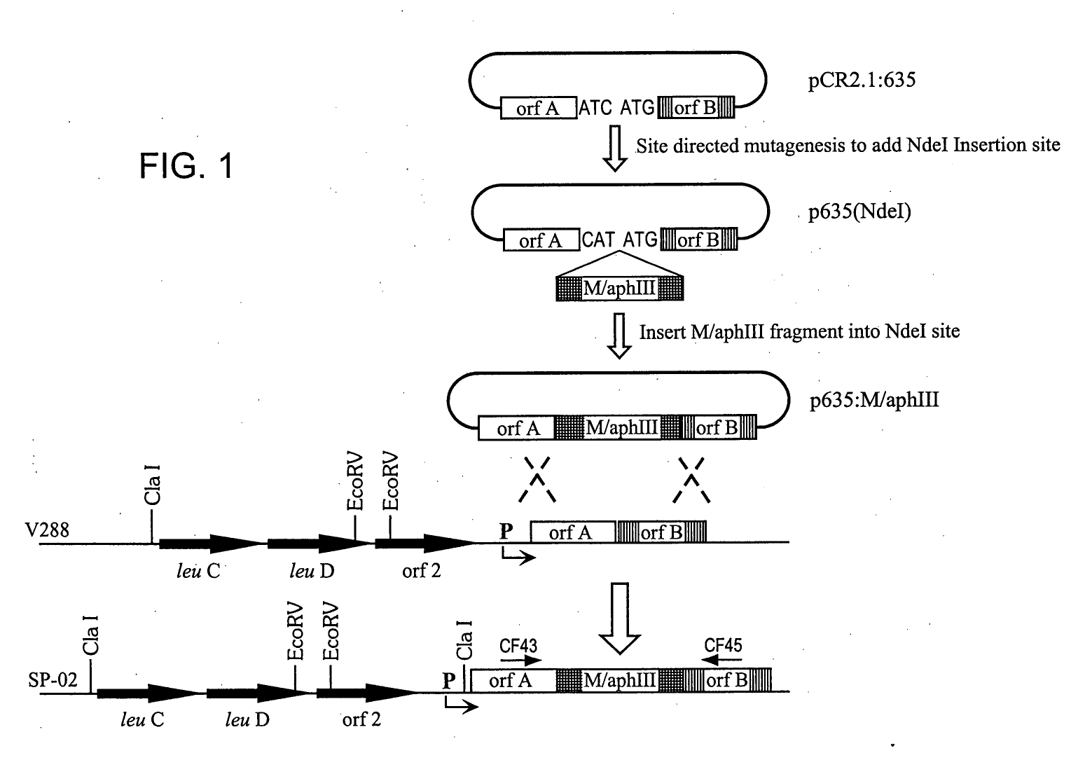

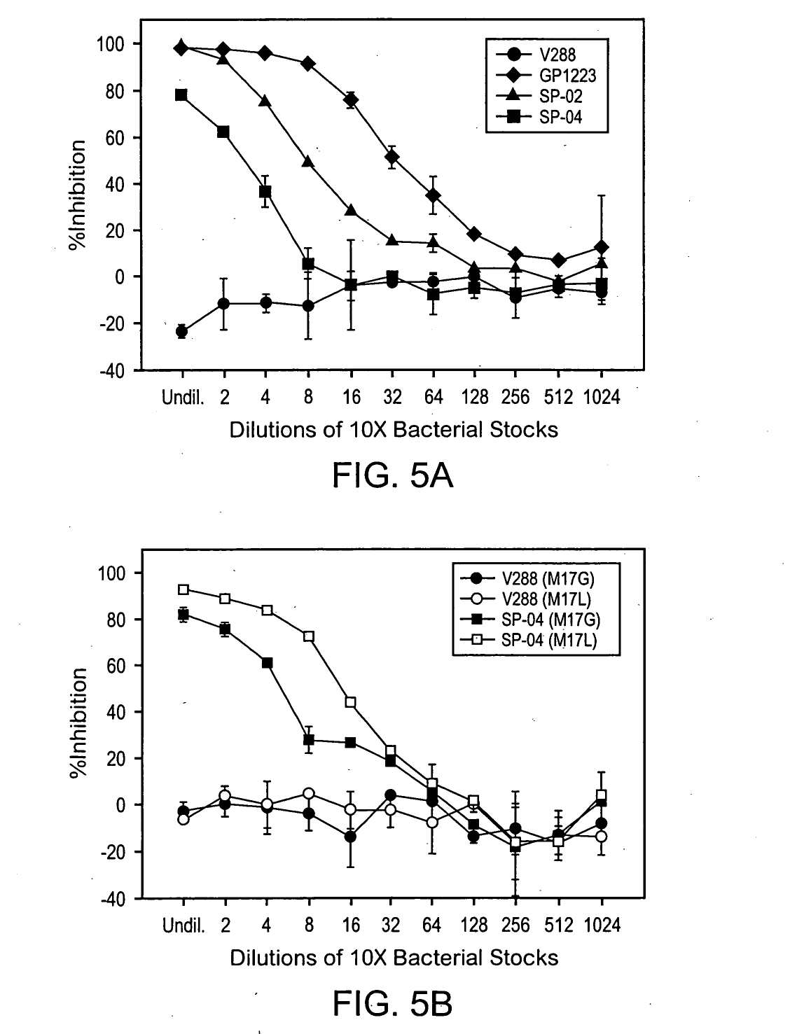

As noted above, several systems have been developed for expressing heterologous proteins in nonpathogenic oral streptococci, such as S. gordonii (9, 16). Pozzi and coworkers developed a chromosomal insertion site in S. gordonii for expressing protein (11). In making the genetically engineered streptococcal recipient strain, the chromosome has undergone some genetic rearrangement and duplication (3). The present study was undertaken to identify additional chromosomal insertion sites in the wild type S. gordonii genetic background for use without significant disruption of the chromosome that might have deleterious effects on the phenotype of derived recombinants which could compromise their eventual use as vaccines. The work done by Franke and Hruby (3) provides some insight into the promoter driving the recombinant genes and the surrounding area in the parental strain. We have taken advantage of this new genomic information to design plasmids that allow insertion of heterologous genes between orfA and orfB (FIG. 1). This allows for a clean and stable chromosomal insertion site that does not disrupt any other loci. Protein expression from this locus was achieved; albeit at about a 7 fold lower level than insertion directly behind the promoter (FIG. 5A). Homology searches to identify this operon and promoter have not identified any known functions, but with further studies this operon may provide several other intergenic insertion sites that provide high levels of expression or that are possibly inducible.

Others have attempted to develop systems to express and over-express heterologous proteins in L. lactis (8, 18, 20). One well-studied pathway in L. lactis is the catabolism of lactose driven by the lac operon. This operon has a divergently transcribed repressor gene (lacR) upstream of the other lac genes (20). Expression of the lac genes has been shown to be induced by growth on lactose (8). Payne et al (8) inserted heterologous genes into the lacG orf, but the lac phenotype was not affected. The present inventors have now identified a portion of the lac operon in S. gordonii and express a heterologous gene inserted within the lacG orf. Protein expression levels from the lacG site were lower than expression from the intergenic “635” site (FIG. 5A), but clearly inducible by the presence of lactose in the growth media (FIG. 5B). This provides a second clean and stable insertion site that can be regulated simply by lactose concentration. Further studies would need to be done to see how controllable this system is and to what level, both in vitro and in vivo. This may be of particular importance when using the gram-positive protein expression system, SPEX, to express proteins in vitro whose activity may be deleterious to bacterial growth (e.g. proteases). Likewise the ability to induce foreign gene expression on demand may provide a mechanism for pulsed delivery of antigen to the immune system to maximize the protective immune response without induction of tolerance.

Preferred insertion sites (restriction enzyme sites) for use in the present invention include Ndel, BamHl, BgIII, Clal, EcoRI, EcoRV, HindIII, Hpal, Kpnl, Pvull, Pstl, Sacl, Sall, Scal, Spel, Sphl, Stul, Xbal, and Xhol. Preferred selectable markers for use in the present invention will confer antibiotic resistance, e.g., resistance to kanamycin, erythromycin, spectinomycin, and/or tetracycline. Particularly preferred selectable markers include the kanamycin resistance gene aphIII, the erythromycin resistance genes ermC, and ermAM, the spectinomycin resistance gene aadA, and the tetracycline resistance genes tetM and tetO.

The constructs of the present invention are useful for introducing heterologous genes into any gram-positive bacterium. Suitable gram-positive bacteria include Streptococcus gordonii, Streptococcus pyogenes, Streptococcus mutans, Streptococcus epidermidis, Streptococcus pneumoniae, Lactococcus lactis, Lactobacillis helveticus, Lactobacillis paracasei, Enterococcus faecalis, Staphylococcus aureus, Group B streptococci, Group G streptococci, Peptostreptococcus magnus, Streptococcus dysgalactiae, Streptococcus suis, Streptococcus sobrinus, Listeria monocytogenes, Actinomyces viscosis, Actinomyces naeslundii, Streptococcus zooepidemicus, Streptococcus equisimilis, Streptococcus sobrinus, Bacillus licheniformis, Streptococcus sanguis, and Streptococcus salivarius.

The following examples are presented in order to more fully illustrate the invention. They should in no way be construed, however, as limiting the broad scope of the invention.

### EXAMPLE 1

Materials and Methods

Bacteriological methods. Strains, plasmids, and primers used in this Example are listed in Table 1.

TABLE 1

Bacterial strains, plasmids and oligonucleotides

| Strain, plasmid | | Reference |
|---|---|---|
| or oligo | Relevant markers and characteristics | or source |
| Strains | | |
| E. coli | | |
| INVαF | F endA1 recA1 hsdR17(rₖ⁻, mₖ⁺) supE44 thi-1 gyrA96 | Invitrogen |
| | relA1Ö80lacZÄM15 Ä(lacZYA-argF)U169 | |
| XL1 Blue | recA1 endA1 gyrA96 thi-1 hsdR17 supE44 relA1 lac | Stratagene |
| | [F proAB lacI^{q}ZÄM15 Tn10 (Tet^{r})]^{c} | |
| S. gordonii | | |
| V288 | Wild Type (ATCC 35105) | ATCC |
| GP1223 | M protein recombinant strain that expresses M6 protein (S. | G. Pozzi |
| | pyogenes) residues 1 to 16 fused to residues 222-441 and | |
| | contains an aphIII gene, Km^{r} | |
| SP-02 | M protein recombinant strain, p635:M/aphIII in V288, Km^{r} | This work |
| SP-04 | M protein recombinant strain, pLacG:M/aphIII in V288, Km^{r} | This work |
| 635/ermC | M protein recombinant strain, p635/ermC in V288, Em^{r} | This work |
| LacG/ermC | M protein recombinant strain, pLacG/ermC in V288, Em^{r} | This work |
| Plasmids | | |
| pCR2.1 | Km^{r}, Amp^{r} | Invitrogen |
| pCR2.1:635 | 1.1-kb PCR-amplified 6-35 walk from V288 cloned into | This work |
| | pCR2.1 at EcoRI, Amp^{r} | |
| p635(Ndel) | Ndel site incorporated in between orf 1 and orf 2 in | This work |
| | pCR2.1:635, Amp^{r} | |
| pCR2.1:6-86 | 1733 bp PCR amplified 6-86 walk containing most of the | This work |
| | lacG gene and part of the lacE gene from V288 cloned into | |
| | pCR2.1 at EcoRI, Amp^{r} | |
| pLacG | Derivative of pCR2.1 carrying 1.7-kb lacE/G cassette with | This work |
| | Ndel incorporated within the lacG ORF, Amp^{r} | |
| p635(Ndel) | | This work |
| derivatives | | |
| p635/ermC | 1.2-kb ermC fragment from pSMB104 cloned into Ndel site, | This work |
| | Amp^{r} | |
| p635:M/aphIII | 2.7-kb M/aphIII fragment from GP1223 cloned into Ndel site, | This work |
| | Amp^{r} | |
| pLacG | | |
| derivatives | | |
| pLacG/ermC | 1.2-kb ermC fragment from pSMB104 cloned into Ndel site, | This work |
| | Amp^{r} | |
| pLacG:M/aphIII | 2.7-kb M/aphIII fragment from GP1223 cloned into Ndel site, | This work |
| | Amp^{r} | |
| Oligonucleotides | | |
| CF4 | 5′-AATAGGGCTCGAGCGGC-3′ | 23 |
| CF5 | 5′-GGATCCTAATACGACTCACTATAGGGC-3′ | 23 |
| CF6 | 5′-AATAGGGCTCGAGCGGC-3′ | 23 |
| CF7 | 5′-ACCTGCCC-(c3-lcaa-CPG spacer) | 3 |
| CF35 | 5′-CGATTCGACATAGAAATAAATTGGAG-3′ | 3 |
| CF43 | 5′-GTTTGGTGACCTATAGTCAGTG-3′ | 3 |
| CF45 | 5′-TGGATGGCATGAATGTATAGAT-3′ | 3 |
| TB59 | 5′-AAAGAAGCATAACATATGTCAAAACAAG-3′ | This work |
| TB85 | 5′-ACACTTCATCACTTTGATACCCCAGA-3 | This work |
| TB86 | 5′-CCATTTGACCATGAGAAGACATCCATC-3′ | This work |
| TB95 | 5′-AAATCTCCATTTGAATGAAGTGCCTCTGGGG-3′ | This work |
| TB96 | 5′-GTCCACAAAGTGCTCAATATTATCCCGATTGAG-3′ | This work |
| TB100 | 5′-AGGGCGTCAGAGAATCTCCAACCCATATACC-3′ | This work |
| TB103 | 5′-GGAATTCCATATGCGGATAATAAATATATATAAACG-3′ | This work |
| TB104 | 5′GGAATTCCATATGCGATTCACAAAAAATAGGCACACG3′ | This work |
| TB107 | 5′-GCAGGAGTGGACGAAGAAGCTCC-3′ | This work |
| TB117 | 5′-GGATCCCATATGTAAGGAGCATAAAAATGGC-3′ | This work |

Escherichia coli strains were grown in Luria-Bertani broth or on Luria-Bertani medium containing 1.5% agar. S. gordonii was plated on or cultured in Brain-Heart infusion (BHI, Difco) with or without 1.5% agar respectively. M17 (Difco) supplemented with 2% glucose (M17G) or 2% lactose (M17L) was also used for culturing S. gordonii stains for the induction experiments. Ampicillin was added at 50 μg/ml for E. coli, erythromycin was used at 5 μg/ml and kanamycin was used at 500 μg/ml for S. gordonii. Frozen cells of naturally competent S. gordonii V288 were prepared and transformed as previously described (10). Standard procedures were used for gene fusions and mutagenesis in E. coli vectors (6). Chromosomal DNA from S. gordonii strains was prepared as described previously (1).

Construction of 635 insertional mutants. An 1153 bp DNA fragment consisting of orf A and orf B, was amplified by PCR with primers CF6 and CF35. The amplified product was purified and cloned into pCR2.1-TOPO vector to yield the plasmid pCR2.1:635. A Ndel site was created, with primer TB59, between orf A and orf B in pCR2.1:635 using the Quick Change™ site directed mutagenesis kit (Stratagene) yielding p635(Ndel) (FIG. 1). The erythromycin gene from pSMB104 was amplified using primers TB103 and TB104 and inserted into the Ndel site of p635(Ndel) yielding p635/ermC. S. gordonii V288 were transformed with p635/ermC and generated erythromycin resistant strain 635/ermC. A 2.7 Kb M/aphIII fragment, containing the emm6 gene (12) fused to the aphIII gene, was amplified from S. gordonii GP1223 with primers TB117 and TB104. The amplified product was purified and digested with Ndel and cloned into the Ndel site in p635(Ndel) yielding p635:M/aphIII (FIG. 1). Competent cells of S. gordonii V288 were transformed with p635:M/aphIII and generated kanamycin resistant strain SP-02 (FIG. 1). 635/ermC and SP-02 were verified by PCR analysis across the plasmid-chromosome junction with the primer pair CF43 and CF45 and by southern blot analysis.

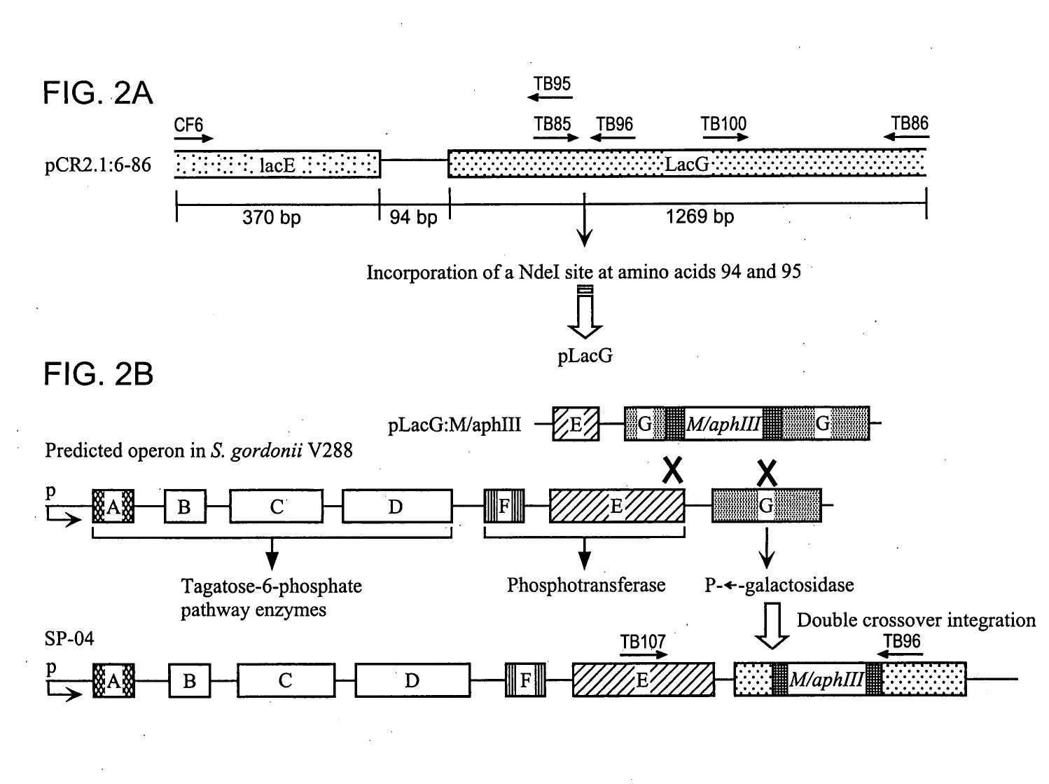

Construction of LacG knockout mutants. The lac operon sequence from S. mutans (15) was used to run a homology search against the S. pyogenes sequence database (14). Primers TB85 and TB86 were designed based on the highly conserved regions within the lacG orf. A 958 bp lacG fragment was PCR amplified using TB85 and TB86 from chromosomal DNA prepared from SP204(1-1) (FIG. 2A). DNA upstream and downstream of this 958 bp region was cloned by chromosomal walking as described previously (17). Briefly, chromosomal DNA from V288 was digested with EcoRV, Pvull or Scal and ligated with adapter primers CF4 and CF7. This adaptor ligated DNA was used as template for PCR using adaptor primers (CF5 and CF6) and gene specific primers (TB95, TB96 and TB100) (FIG. 2A). This previously unpublished sequence from the lac operon in S. gordonii was submitted to GenBank (Accession No. AF210773). A cloned 1733 bp region containing most of the lacG gene and part of the lacE gene in pCR2.1:6-86 (FIG. 2A) was mutagenized by site directed mutagenesis (Stratagene) and a Ndel site was introduced within the lacG orf between amino acids 94 and 95 yielding pLacG. The erythromycin gene from pSMB104 was inserted into the Ndel site in pLacG yielding pLacG/ermC. S. gordonii V288 was transformed with placG/ermC and generated the erythromycin resistant knockout strain LacG/ermC. A 2.7 kb M/aphIII fragment was amplified from S. gordonii GP1223 with primers TB117 and TB104. The amplified product was purified and digested with Ndel and cloned into the Ndel site in pLacG yielding pLacG:M/aphIII (FIG. 2B). Competent cells of S. gordonii V288 were transformed with pLacG:M/aphIII and generated erythromycin resistant strain SP-04 (FIG. 2B). LacG/ermC and SP-04 were verified by PCR analysis across the plasmid-chromosome junction with the primer pair TB107 and TB96 and by southern blot analysis.

Immunological Assays

Streak blot analysis. S. gordonii transformants were streaked on the surface of BHI plates by toothpick transfer of colonies from the selection plates. Each plate contained the transformants, an M6+ strain (GP1223) and an M6− strain (V288) for controls. Streak blot was performed as previously described (9), using monoclonal antibody (mAb) 10F5 (2), raised against the recombinant M6 protein purified from E. coli.

Western blot analysis. The streptococcal strains were grown to late stationary phase in BHI. 300 μl of culture was pelleted by centrifugation in 1.5 ml microfuge tubes. The culture supernatant was acetone-precipitated and the pellet was resuspended in SDS sample buffer. The samples were run on a 4-12% Bis-Tris gel and transferred to a Millipore Immobilon-P transfer membrane. Western blotting was performed as previously described (2) using mAb 10F5.

Competition ELISA. Streptococcal overnight cultures were back-diluted 1:100 in BHI (M17G and M17L media was used for induction studies with the SP-04 strain) containing the appropriate antibiotics and grown to late log (OD650 nm=0.6-0.7). 50 ml of culture was harvested by centrifugation (10,000×g) for 10 min and the cell pellets were resuspended in 25 ml PBS/azide (PBS+0.02% sodium azide). The bacterial suspensions were placed in a 56° C. water bath for 60 minutes to kill the cells. The cells were centrifuged and washed with 25 ml PBS/azide. The cell pellets were resuspended in 10 ml PBS/azide and the OD650nm was adjusted to 1.0 with PBS/azide. 10 ml of adjusted suspension was centrifuged and 9 ml of supernatant was removed by pipet. The pellet was resuspended with the remaining supernatant. Strain preparations were stored at 4° C. for up to 1 week. The resulting cell suspensions were used to compete for the binding of mAb 10F5 to recombinant M6 protein in competition ELISAs, as described by Jones et al (4, 5).

Southern blot analysis. Gene-specific probes were obtained after appropriate digestion of DNA from plasmids pCR2.1:635 (635 probe, 1153 bp), pLacG (LacG probe, 1791 bp) and p635:M/aphIII (M/aphIII probe, 2702 bp). DNA probes were gel isolated, cleaned and labeled with the Redivue 32P dCTP rediprime II random prime labeling system (Amersham). Chromosomal DNA from V288 and SP-02 was digested with Clal/Smal and DNA from V288 and SP-04 was digested with Smal/Xbal. DNA fragments were separated on a 0.7% agarose gel and transferred to Zeta-Probe GT Genomic blotting membranes (BIO-RAD) by capillary transfer. Membranes were hybridized to specific DNA probes labeled with 32P as described above. Hybridization conditions were as recommended by the manufacturer. Blots were exposed to X-OMAT AR film (Kodak) at −70° C. for 1 hour and developed in a HOPE Micro-Max developer.

Results

Construction of an intergenic mutant. Genomic analysis of recombinant S. gordonii GP1223 and the parent V288 strain revealed that the inserted foreign sequences (recombined into GP1223 chromosome) were being driven by a promoter normally located in front of two unknown open reading frames, orf A and orf B, which are just downstream of the leucine operon (3). A Ndel site was introduced between orf A and orf B in p635 (FIG. 1) to serve as an insertion site between the two genes. First, an erythromycin gene was inserted and the resulting plasmid p635:ermC was transformed into V288. Since p635:ermC has no gram-positive origin of replication it cannot replicate in S. gordonii and was forced to integrate into the chromosome via homologous recombination and yielded erythromycin resistant colonies. PCR analysis of this double cross-over mutant 635/ermC with the primer pair CF43-CF45 produced a product that was 1.2 kb larger than wild type V288 due to insertion of the erythromycin gene.

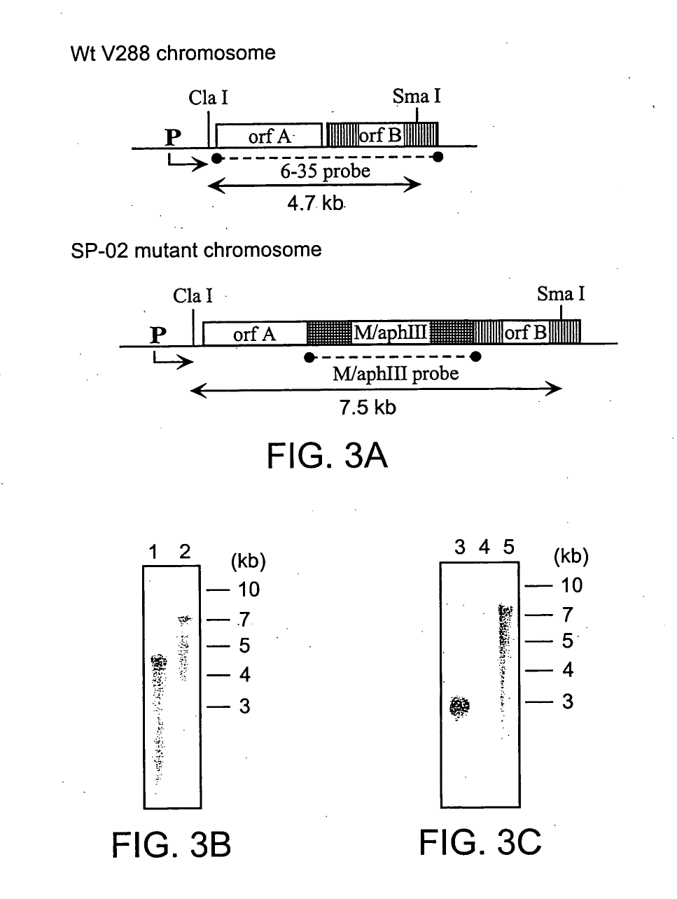

The emm6 (12) gene fused to the aphIII gene (M/aphIII) was then inserted into p635 at the engineered Ndel site and the resulting plasmid p635:M/aphIII was transformed into V288 yielding kanamycin resistant colonies. This recombinant, SP-02, containing the M/aphIII fusion between orf A and orf B was verified by PCR using the primer pair CF43-CF45. SP-02 produced a product that was 2.7 kb larger than wild type V288 due to insertion of the M/aphIII cassette (data not shown). Southern blot analysis on SP-02 genomic DNA, restricted with Clal and Smal, using a portion of the 635 sequence and the M/aphIII sequence as labeled probes showed an intergenic insertion event had occurred. The 635 probe reacted with a 4.7 kb band in V288 (also restricted with Clal and Smal) and a 7.5 kb band in SP-02 which is a difference of 2.7 kb, the size of the insert (FIG. 3B), suggesting that it is a double cross-over mutant. The M/aphIII probe did not react with V288 DNA, which does not have the M/aphIII gene, and reacted with a 7.5 kb band in SP-02 as expected (FIG. 3C). M6 surface protein expression was demonstrated in SP-02 by streak blot (data not shown), and competition ELISA (FIG. 5A). The ELISA showed that the expression levels of SP-02 were about 3 fold lower than that of GP1223, which has the M protein gene inserted directly behind the promoter. Wild type strain V288 was used as a negative control in the competition ELISAs. The expected size of the M6 protein (28 KDa) was verified by western blot.

Identification of the LacG operon in S. gordonii. In Lactococcus lactis the catabolism of lactose is initiated by a phosphoenolpyruvate-dependent phosphotransferase system. The genes that encode the phospho-β-galactosidase (lacG), the lactose-specific components of the phosphotransferase system (lacE and lacF) and the tagatose 6-phosphate pathway enzymes (lacA, lacB, lacC and lacD) are located in the same operon and are transcribed from the same promoter (FIG. 2B) (8,20). This same operon configuration has been found in S. mutans (15). Based on a homology search between the S. mutans lac operon sequence and the S. pyogenes sequence database (14), a similar operon was found in S. pyogenes. Primers TB85 and TB86 were designed to the most highly conserved regions within the lacG gene. These primers produced a 958 bp PCR product from the S. gordonii genomic DNA (FIG. 2A) that was approximately 80% identical to the lacG sequence from both S. mutans and S. pyogenes. Chromosomal walks in the S. gordonii chromosome produced a portion of the lacE gene upstream and the rest of the lacG gene downstream. The C-terminal end of the lacE gene and most of the lacG gene were cloned into pCR2.1 and an Ndel site was incorporated between amino acids 94 and 95 of lacG creating pLacG (FIG. 2A).

Construction of an intragenic LacG mutant. It was established by Payne et al (8) that foreign genes could be inserted into the lacG gene orf for chromosomal expression. This intragenic insertion event inactivates the lacG gene, but has no obvious deleterious affects on the lac phenotype of the derived recombinant. The ability to insertionally inactivate the lacG gene is thought to be possible because there is a separate enzyme present in the strain that has secondary phospho-β-galactosidase activity (8, 19). The lac operon promoter has been used for controlled expression for chromosomally integrated genes (8). The pLacG plasmid serves as a way to insert genes into the chromosome of S. gordonii within the lacG gene. First the erythromycin gene was inserted at Ndel and the resulting plasmid pLacG:ermC was transformed into S. gordonii V288. Chromosomal insertion of an erythromycin resistant transformant was verified by PCR using the primer pair TB107-TB96 (data not shown) and produced a product that was 1.2 Kb larger than wild type due to insertion of the erythromycin gene. This mutant was called LacG:ermC.

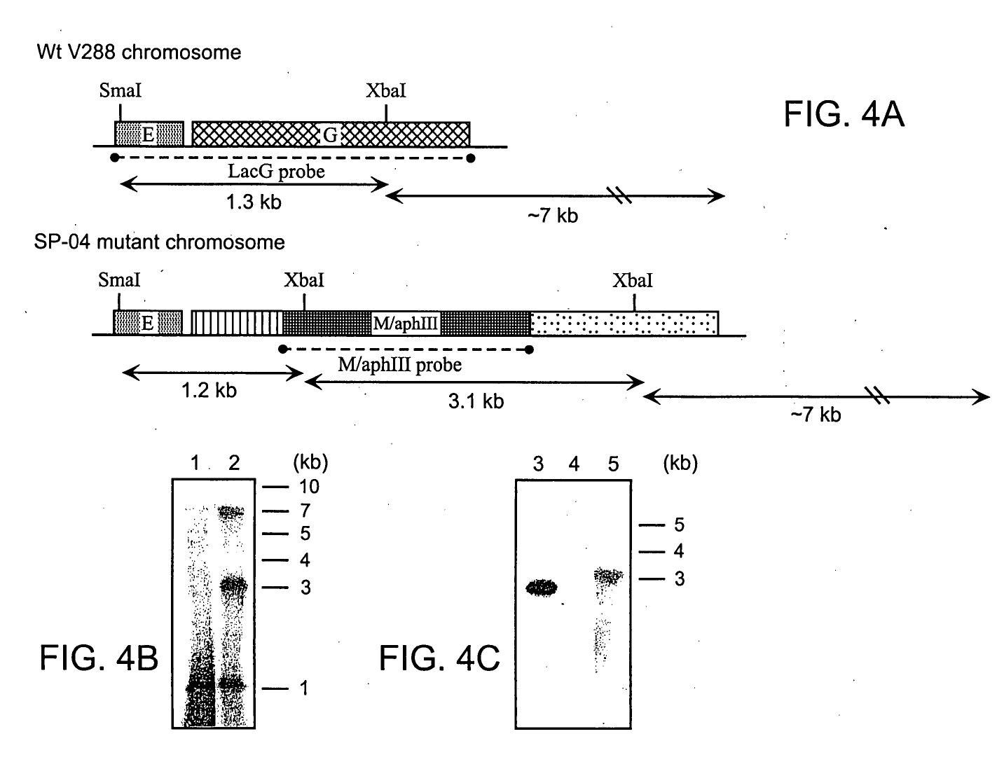

Next, the M/aphIII fusion cassette was inserted into pLacG to yield plasmid pLacG:M/aphIII. S. gordonii V288 was transformed with pLacG:M/aphIII and transformants were selected on BHI containing kanamycin. The resulting double cross-over LacG knockout mutant SP-04 (FIG. 2B) was verified by PCR using the primer pair TB107-TB96 and produced a product that was 2.7 kb larger than the wild type product due to insertion of the M/aphIII cassette (data not shown). SP-04 genomic DNA restricted with Smal and Xbal was also verified by southern blot analysis using a lacE/G labeled probe and a M/aphIII labeled probe. The lacE/G probe reacted with a 1.3 kb band and a 7 kb band in V288 (also restricted with Smal and Xbal) and three bands (1.2 kb, 3.1 kb, and 7 kb) in SP-04 as expected for a double cross over mutant in this locus (FIG. 4B). The M/aphII probe did not react with V288 since it does not contain the M/aphIII fusion and it reacted with a 3.1 kb band in SP-04 as expected for insertion into the lacG gene (FIG. 4C). To verify that the M6 protein was expressed on the surface of SP-04, a streak blot was performed on colonies lifted from a BHI plate (data not shown), and a competition ELISA was performed using anti-M6 monoclonal antibodies (FIG. 5A). Wild type strain V288 was used as a negative control in the competition ELISA's. The results of the ELISA showed that the anti-M6 antibody reacted with cell surface-expressed M6 protein of SP-04 (FIG. 5A) at a lower level than GP1223. M6 protein expression from SP-04 was about 4 fold below GP1223 and SP-02 was about 3 fold lower than SP-04 (FIG. 5A). Expression of the lactose operon has been shown to be under the control of a regulator protein produced by the divergently transcribed lacR gene (8). With SP-04, a 4 fold increase in the level of M6 expression was obtained when cells were grown in the presence of lactose (M17L) compared to glucose (M17G) (FIG. 5B). Western blot analysis of supernatant from SP-04 showed the correct 28 KDa band.

### EXAMPLE 2

### Genomic Organization of Recombinant Streptococcus gordonii Strain Expressing the C-repeat Region of Streptococcus pyogenes M6 Protein

Materials and Methods

Bacterial strains, plasmids, and oligonucleotides. The bacterial strains, plasmids, and oligonucleotides used or relevant to this study are listed in TABLE 2. Escherichia coli strains were grown in Luria-Bertani broth and S. gordonii strains in brain heart infusion broth (BHI; Difco Laboratories, Detroit, Mich.). All bacterial cultures were incubated at 37° C. Kanamycin (500 mg/ml) and streptomycin (500 mg/ml) were used whenever required for S. gordonii strains and ampicillin (50 mg/ml) for the selection and growth of E. coli strain INVαF′ containing the plasmid pCR2.1 clones. The oligonucleotides, described in TABLE 1 were synthesized by either the Central Services Laboratory (Oregon State University) or Gibco-BRL Laboratories.

TABLE 2

Bacterial strains, plasmids, and oligonucleotides

| | | |
|---|---|---|
| Strain, plasmid, or | | Reference or |
| oligonucleotide | Relevant markers and characteristics | source |

| | | |
|---|---|---|
| Strains | | |
| E. coli | | |
| INVαF′ | F′ endA1 recA1 hsdR17(rₖ⁻, mₖ⁺) supE44 thi-1 | Invitrogen |
| | gyrA96 relA1ö80lacZÄM15 Ä(lacZYA-argF)U169 | |
| S. gordonii Challis | | |
| V288 | Wild-type (ATCC 35105) | ATCC |
| GP204 | Spontaneous Sm^{r} mutant of V288 | Pozzi et al., |
| | | 1988 |
| GP230 | Recombinant strain contains the emm6 gene (S. | Pozzi et al., |
| | pyogenes) and an ermC gene, Em^{r}, parent strain | 1992 |
| | (V288) | |
| GP251 | Recombinant recipient strain contains the cat | Oggioni et al., |
| | gene flanked by 145 bp of emm6 gene and 202 | 1996 |
| | bp of ermC gene, Cm^{r}, parent strain (GP230) | |
| GP1214 | Recombinant strain that expresses M6 protein | Oggioni et al, |
| | (S. pyogenes) residues 1 to 16 fused to residues | 1994 |
| | 222-441 and contains an ermC gene, Em^{r}, | |
| | parent strain (GP251) | |
| GP1218 | Recombinant strain that expresses M6 protein | Oggioni et al, |
| | (S. pyogenes) residues 1 to 16 fused to residues | 1994 |
| | 222-441 and contains an aphIII gene, Km^{r}, | |
| | parent strain (GP1214) | |
| GP1223 | Recombinant strain that expresses M6 protein | Oggioni et al, |
| | (S. pyogenes) residues 1 to 16 fused to residues | 1994 |
| | 222-441 and contains an aphIII gene, Km^{r}, and | |
| | has been converted to Sm^{r}, parent strain | |
| | (GP1218) | |
| Plasmids | | |
| pCR2.1 | Km^{r}, Amp^{r} | Invitrogen |
| pSMB104 | Contains the sequences encoding the CRR of | Oggioni et al, |
| | M6 protein (S. pyogenes) residues 1 to 16 fused | 1994 |
| | to residues 222-441 in tandem with an MspI/ClaI | |
| | fragment of pE194 () encoding ermC cloned into | |
| | pBluesccipt SK-. | |
| Oligonucleotides | | |
| CF4 | 5′-CTAATACGACTCACTATAGGGCTCGAGCG | Siebert et al, |
| | GCCGCCC GGGCAGGT-3′; Adaptor | 1995 |
| CF5 | 5′-GGATCCTAATACGACTCACTATAGGGC-3′; | Siebert et al, |
| | AP1 | 1995 |
| CF6 | 5′-AATAGGGCTCGAGCGGC-3′; AP2, SEQ | Siebert et al, |
| | | 1995 |
| CF7 | 5′-ACCTGCCC-(C3-lcaa-CPG spacer); AP1 | This study |
| CF8 | 5′-TCTAGAGGTACCTTCTCGTGCTTTGTCCGG- | This study |
| | 3′; PCR (GP1223) | |
| CF9 | 5′-TACCGTCCCCCTAGGAAACACTCTTGCAC- | This study |
| | 3′; SEQ, PCR (GP1223) | |
| CF10 | 5′-TGACTTACTGGGGATCAAGCCTGATTGGG | This study |
| | AG-3′; PCR (GP1223) | |
| CF11 | 5′-AAGTACATCCGCAACTGTCCATACTCTGAT | This study |
| | G-3′; PCR (GP1223) | |
| CF14 | 5′-GTTTTTCGTGTGCCTATTTTTTGTG-3′, SEQ | This study |
| | 1223 | |
| CF15 | 5′-GAGCGCATCGAAAATGCTGTTG-3′; SEQ, | This study |
| | PCR (GP204 | |
| CF16 | 5′-CTCAGTGTAAAGAGGAAATCC-3′; SEQ | This study |
| CF17 | 5′-GAGTTTCAATGGTCTTGTCTGG-3′; SEQ, | This study |
| | PCR (GP204, GP1223) | |
| CF18 | 5′-CTTGAAAAGCCTGAGGGCTGGTTAC-3′; | This study |
| | SEQ, PCR (GP204) | |
| CF19 | 5′-CTTGACCTTTGGTACCTTTGAC-3′; SEQ | This study |
| CF20 | 5′-GATAGTCACACGGCTACTCACG-3′; SEQ | This study |
| CF21 | 5′-CGTGAGTAGCCGTGTGACTATC-3′; SEQ | This study |
| CF22 | 5′-GTCCATAGAGTTTGGATCCAAG-3′; SEQ | This study |
| CF23 | 5′-GTCAAAGGTACCAAAGGTCAAG-3′; SEQ | This study |
| CF24 | 5′-CCAGAAATTCGCGATATGAAC-3′; SEQ | This study |
| CF25 | 5′-GAATGAATCCAGATAAGGTGC-3′; SEQ | This study |
| CF26 | 5′-GATATCTTCAACTCATGGGATTAC-3′; SEQ, | This study |
| | PCR (GP204) | |
| CF27 | 5′-CAAGATTCTCACCAGTTTTATG-3′; SEQ | This study |
| CF28 | 5′-GCTGCGATGCTTATGATTACC-3′; SEQ | This study |
| CF29 | 5′-GCTACCAATGCTGACAATAG-3′; SEQ | This study |
| CF31 | 5′-CCTAAGCAGTTTCTCAAGTTG-3′; SEQ | This study |
| CF32 | 5′-CATGTTGCCTATCGTCCAGC-3′; SEQ PCR | This study |
| | (GP204, GP1223) | |
| CF35 | 5′-CGATTCGACATAGAAATAAATTGGAG-3′; | This study |
| | SEQ, PCR (GP204) | |
| CF36 | 5′-CTATAGTCAGTGTGGTTTAGACAAGC-3′; | This study |
| | SEQ | |
| CF39 | 5′-GATTATGCTGAATCAAATAGTC-3′, SEQ | This study |
| CF40 | 5′-GAGCACGATAGTAGTCAATCAC-3′; SEQ | This study |
| CF41 | 5′-CAATTTTTGACTGATACGATGGC-3′; SEQ | This study |
| CF42 | 5′-CTGTTCTTCCAACTTTTTCAGC-3′; SEQ | This study |
| CF43 | 5′-GTTTGGTGACCTATAGTCAGTG-3′; SEQ | This study |
| CF44 | 5′-ATCTATACATTCATGCCATCCA-3′; SEQ | This study |
| CF45 | 5′-TGGATGGCATGAATGTATAGAT-3′; SEQ | This study |

Chromosomal walks. Chromosomal DNA was prepared from GP204 and GP1223 cells lysed with lysozyme and sodium dodecyl sulfate at pH 8.0 followed by three cycles of freezing and thawing and purified by phenol extraction. Chromosomal walks from a known region to an unknown region in uncloned genomic DNA were accomplished using an improved adaptor ligation PCR method (Siebert, P. D., et al. 1995. Nucl. Acids Res. 23:1087-1088).

Nucleotide sequence methods and analysis. PCR products of chromosomal walks were either sequenced directly or cloned into a TA-cloning vector pCR2.1 (Invitrogen) prior to sequence determination. Sequence determinations were performed at the Central Services Laboratory of the Center for Gene Research and Biotechnology (Oregon State University) using the dideoxy chain termination method. The M13 reverse sequencing primer and the T7 promoter primer were utilized to determine the sequence of PCR inserts cloned into pCR2.1, as well as the specifically designed primers listed in TABLE 2. Sequences were compiled and DNA and amino acid sequences were analyzed using programs developed by the Genetic Computer Group at the University of Wisconsin (Devereux, J., et al. 1984. Nucl. Acids. Res. 12:387-395). The BLAST programs (Altschul, S. F., et al. 1997 Nucl. Acids Res. 25:3389-3402) were used to compare the determined nucleotide sequences to the sequences in the GenBank databases.

Southern blot analysis. S. gordonii chromosomal DNA (1 μg), purified as described above, was digested with restriction endonuclease EcoRV (New England Biolabs; Beverly, Mass.). DNA fragments were separated in 0.8% agarose-Tris-borate-EDTA and then transferred to Nytran Plus (Schleicher and Schuell; Keene, N.H.) membranes. Probe *P1 (including the C-terminal portion of orf2 and the promoter region) was derived by digestion with Clal of the PCR product generated by PCR amplification with primers CF4 and CF9 from GP204 chromosomal template followed by the gel isolation of the 722-bp digestion product. Probe **P2 (encompassing the leuC and leuD ORFs) was obtained by digestion with Clal of the PCR product generated by PCR amplification with primers CF4 and CF11 from a GP204 chromosomal template followed by the gel isolation of the 1894-bp digestion product. Probe ***P3 (a portion of C-repeat region of M6 protein of S. pyogenes) was obtained from by isolation of the 247-bp EcoRI/HindIII digestion product of pSMB104. The probes were labeled and hybridization products visualized using the Rad-Free Psoralin Biotin Probe Labeling and Hybridization Kit (Schleicher and Schuell; Keene, N.H.).

Isolation of total RNA and Northern blot analysis. S. gordonii total RNA was purified as previously described (Shaw, J. H., and D. B. Clewell. 1985. J. Bacteriol. 164:782-796). RNAs (10 mg) were separated in 1% (wt/vol) agarose −2.2 M formaldehyde gels and then transferred to NYTRAN MaxStrength (Schleicher and Schuell; Keene, N.H.) membranes. Probe *P1 (a portion of C-repeat region of M6 protein of S. pyogenes) was obtained from by isolation of the 247-bp EcoRI/HindIII digestion product of pSMB104. Probe *P2 (a portion of the leuB/leuC region of S. gordonii) by isolation of the 976-bp PCR product generated by PCR amplification with primers CF6 and CF18 from a GP204 chromosomal template. The probes were radiolabeled with [α-32P]dCTP, using a random primers DNA labeling kit, Rediprime (Amersham; Picastaway, N.J.) according to the manufactures instructions.

Nucleotide sequence accession numbers. The sequence of (this region) has been assigned GenBank accession nos AF251027, AF251028, and AF251029.

Results

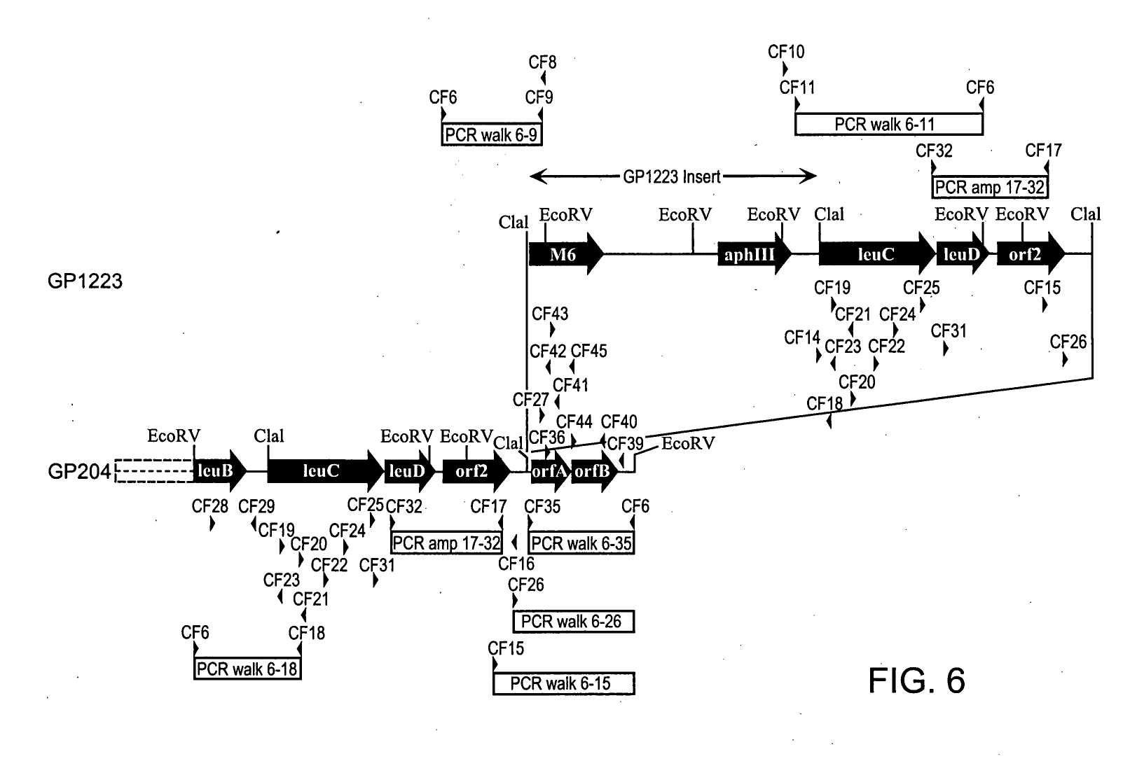

Chromosomal walks upstream and downstream of the GP1223 insert. To determine the chromosomal site of insertion of recombinant strains isolated from recipient S. gordonii strain GP251, a recombinant S. gordonii strain, GP1223, isolated by the targeted insertion of the coding sequence of the CRR of M6 protein of S. pyogenes into this site of GP251 was used as template for directed chromosomal walks upstream and downstream of the GP1223 insert. Chromosomal DNA from S. gordonii strain GP1223 was purified and a special adaptor, CF4 and CF7 (TABLE 2) was ligated to the ends of the DNA fragments generated by digestion of the chromosomal DNA with EcoRV. The adaptor-ligated DNA was used as template for primary and secondary PCR reactions using nested pairs of adaptor primers (CF5, CF6) and a nested pair of specific gene primers (CF8, CF9) to walk upstream of the *Clal M6/aphIII insert or (CF10, CF11) to walk downstream of the insert (TABLE 2 and FIG. 6A). The walk upstream of the insert yielded an 881-bp product designated “PCR walk 6-9” and the walk downstream, a 2175-bp product designated “PCR walk 6-11” as depicted in FIG. 6A. The PCR walk products were sequenced directly commencing with primers CF6, CF9 and CF11, as applicable, and progressing with subsequently designed sequencing primers containing the sequences indicated in TABLE 1 and the positions and polarities illustrated in FIG. 6.

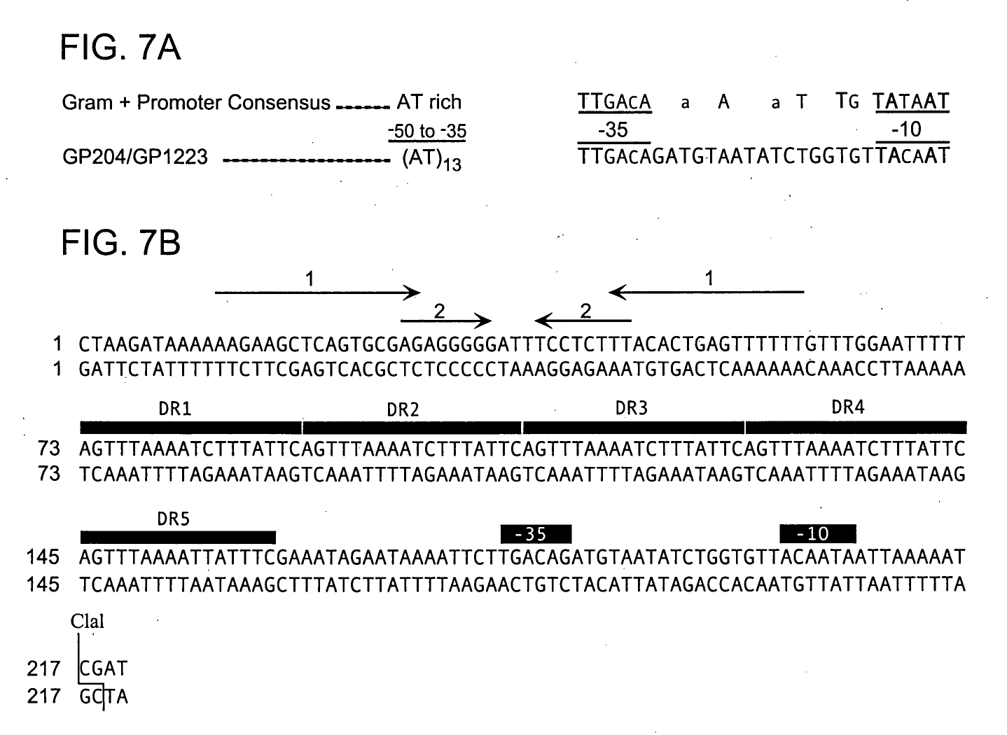

The region upstream of the GP1223 insert contains regulatory signals. Immediately upstream of the GP1223 insert, sequences which conform to the consensus for promoters from gram-positive organisms (DeVos, W. M. 1987 FEMS Microbiol. Rev. 46:281-295; Graves, M. C., and J. C. Rabinowitz. 1986. J. Biol. Chem. 261:11409-11415) were found. The alignment of the gram-positive promoter consensus with the sequence determined from “PCR walk 6-9” is shown in FIG. 7A. This sequence shows the following features in common with the gram-positive promoter consensus: (i) the canonical −35 and −10 sequences; (ii) a spacing between those hexanucleotides of 16 to 18 nucleotides; (iii) the conserved dinucleotide sequence TG, immediately preceding the −10 sequence; and (iv) the AT-rich regions upstream of the −35 sequence (AT-box). Approximately 150 nucleotides upstream of the −35 region, a sequence containing dyad symmetry followed by a stretch of thymidine residues conforms to a prokaryotic factor-independent RNA polymerase terminator sequence (FIG. 7B). Also, a region containing five direct repeats, 4 perfect and 1 imperfect, of 18 nucleotides (AGTTTAAAATCTTTATTC) was observed between the terminator and the promoter sequences (FIG. 7B). Upstream of the terminator sequence, the nucleotide sequence of the 881-bp “PCR walk 6-9” also contained a partial ORF (designated ORF2, see FIG. 6) encoding 169 residues with no apparent functional homologies in the databases at present. The sequence of ORF 2 had not terminated when the walk fragment ended at an EcoRV site to which the walking adaptor was ligated.

The region downstream of the GP1223 insert contains leuC and leuD homologues. Analysis of the nucleotide sequence of “PCR walk 6-11” (FIG. 6A) revealed the presence of two partial ORFs encoding predicted proteins with significant homologies to the large subunit (leuC, pir S35134) and small subunit (leuD, pir E36889) of alpha-isopropylmalate isomerase (EC 4.2.1.33) of Lactococcus lactis, respectively. These gene products are involved in the biosynthesis of the branched-chain amino acids leucine, isoleucine and valine (Godon, J. J., et al. 1992. J. Bacteriol. 174:6580-6589) The ORF encoding the leuC homologue was partial in that it did not contain the initiation codon for the reading frame, but was open from the start of the sequence at the Clal site. The partial leuC ORF of S. gordonii encoded 456 amino acid residues, whereas the complete leuC ORF of L. lactis is 460 residues in size. Nine nucleotides separate the termination codon of the leuC ORF and the initiation codon of the next ORF encoding the leuD homologue. The sequence of the leuD ORF was also partial because the ORF had not terminated when the walk fragment ended at an EcoRV site to which the walking adaptor was ligated. The partial leuD ORF of S. gordonii consisted of 172 residues as compared to the complete leuD ORF (191 residues) of L. lactis.

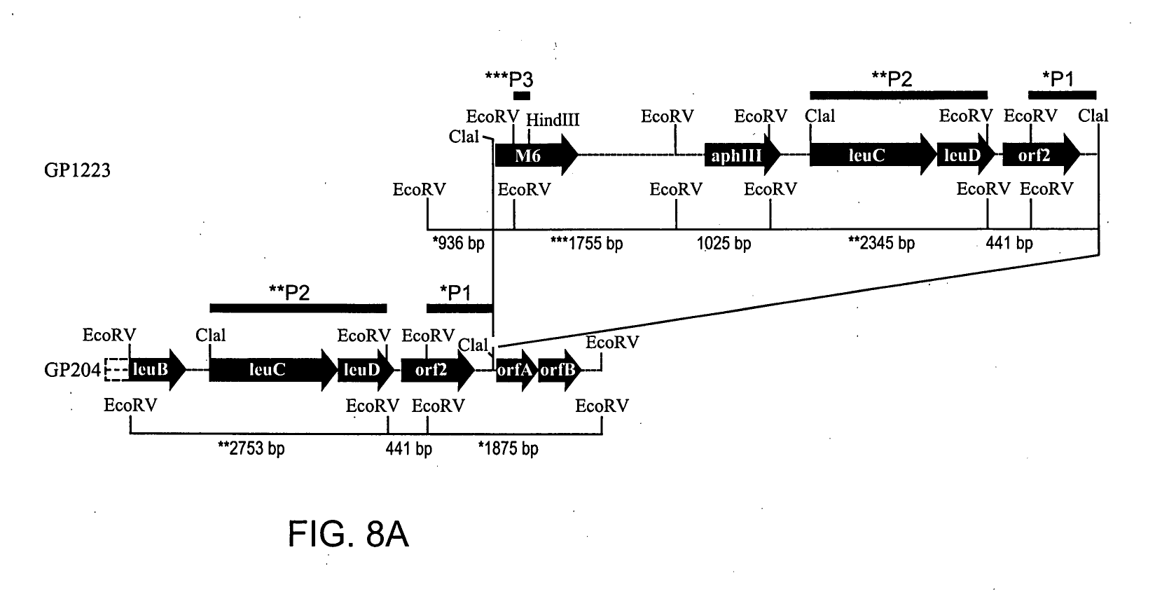

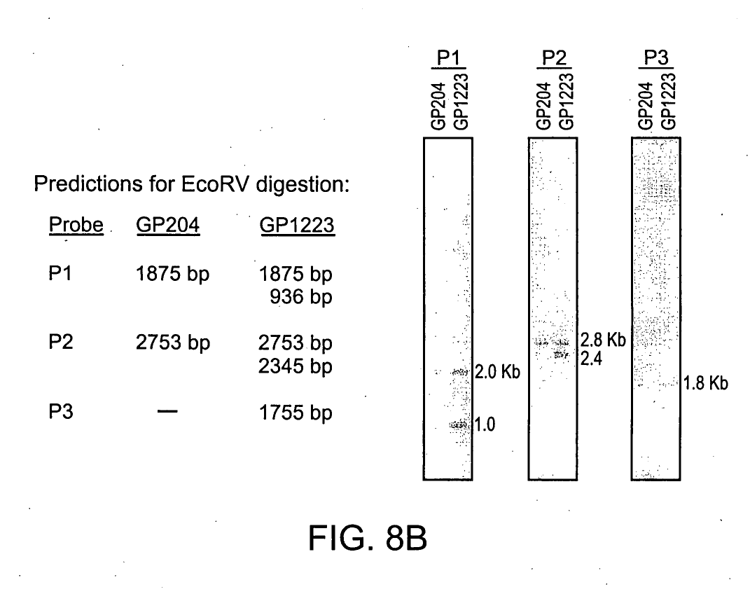

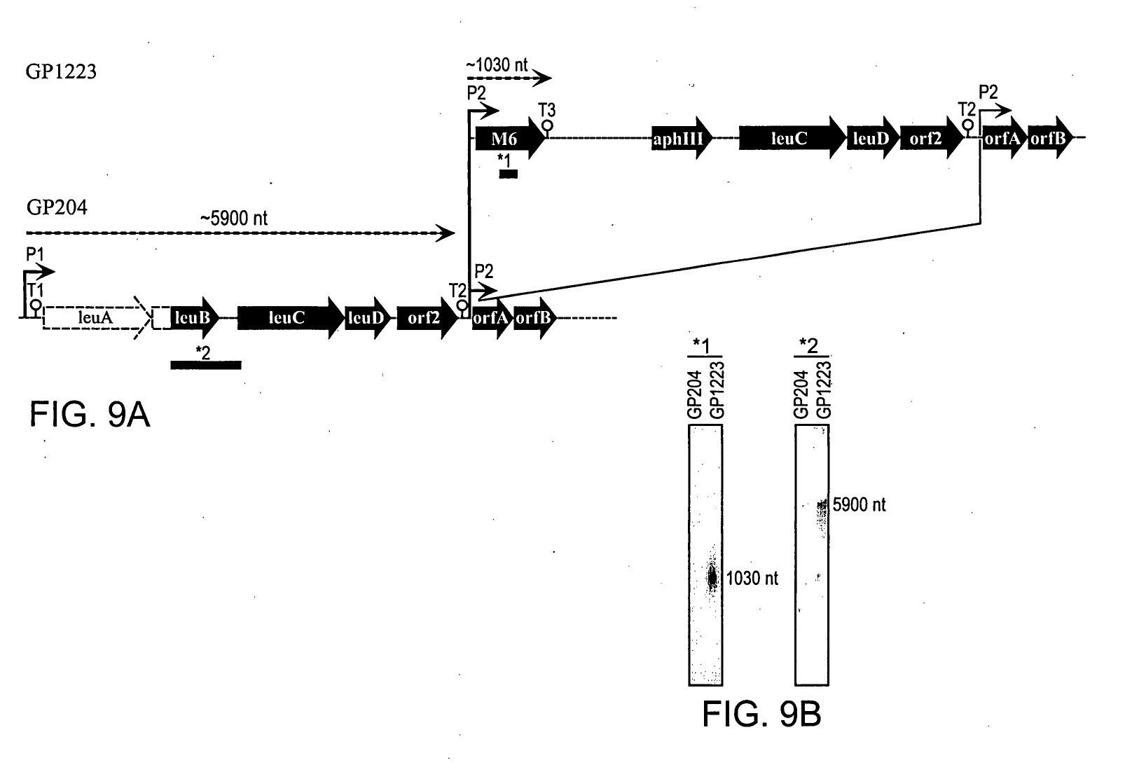

The Clal fragment flanking the GP1223 insert is duplicated. In order to corroborate and extend the structural organization deduced from the genomic walks described above, Southern blot analyses were carried out on chromosomal DNA from S. gordonii strains, GP204 and GP1223. Initially, chromosomal DNA was digested with restriction endonuclease Clal, electrophoretically separated fragments blotted to membranes and probed with radiolabeled DNA fragments obtained from “PCR walk 6-9” and “PCR walk 6-11” digested with Clal. Interestingly, both the probe specific for the upstream PCR walk (6-9) and the probe specific for the downstream PCR walk (6-11) hybridized to fragments that were indistinguishable in size (˜3,000-bp) from both GP204 and GP1223 (data not shown). This result suggested that “PCR walk 6-9” and “PCR walk 6-11” might be contained within the same or a related DNA fragment. In order to determine if an internal EcoRV fragment linked the upstream (6-9) and downstream (6-1 1) PCR fragments on a single Clal fragment, PCR amplification with primers CF17 and CF32 was performed utilizing either GP204 or GP1223 chromosomal DNA as template. As predicted, a ˜930-bp PCR amplification product was produced from both GP204 and GP1223 template DNA (FIG. 6A and 6B) and the nucleotide sequence of these products was determined. The nucleotide sequence of the “PCR amp 17-32” product from both templates was identical and analysis revealed that they encoded the remaining nine residues of the previously determined leuD ORF (for a total leuD ORF of 181 residues). After a gap of 144 nt, a predicted ORF contiguous with the partial ORF2 determined on the sequence of “PCR walk 6-9” added 90 amino acids to the previously determined partial ORF2 of 169 residues yielding a total size for ORF2 of 259 residues. Adding the internal 441 -bp EcoRV fragment, revealed from the sequence analysis of “PCR amp 17-32”, the proposed structure of the 3057-bp Clal fragment present as a single copy in GP204 and duplicated on either side of the M6 insertion site of GP1223 is depicted in FIG. 8A. Corroboration of this proposed genomic structure is demonstrated by the Southern blot analyses shown in FIG. 8B. The predicted EcoRV digestion products and hybridization profiles are in agreement with the proposed genomic structure illustrated in FIG. 8A.

The region upstream of the leuC ORF contains leuB ORF. Once the duplication of the Clal fragment containing the leuC and leuD ORFs was confirmed, it was of interest to determine the nucleotide sequence of the region of the chromosome immediately upstream of the leuC ORF from the parental strain, GP204. Genomic walks upstream of the leuC ORF on parental strain GP204 were performed using specific primer CF18, in combination with adaptor primers CF5, CF6 (FIG. 6B). The resulting PCR product, walk 6-18 (976-bp), was cloned into pCR2.1 and the nucleotide sequence was determined. Analysis of this sequence for ORFs predicted it to encode a partial rightward reading ORF of 193 amino acid residues (FIG. 6B).

Comparison of the leucine operon of S. gordonii to other organisms. Assembly and analysis of the complete nucleotide sequence of the duplicated Clal fragment flanking the recombination insertion site revealed ORF homology and structural organizational homology to the leucine operon of Lactococcus lactis, as well as numerous other gram-positive and gram-negative bacteria. Specifically, the predicted products of translation of two of the three reading frames encoded in this fragment display significant homologies with the large subunit (leuC, pir S35134) and small subunit (leuD, pir E36889) of alpha-isopropylmalate isomerase (EC 4.2.1.33) of Lactococcus lactis. The S. gordonii reading frame with homology to leuC of L. lactis was 67% identical (207 identities over 307 residues) and 81 % positive (250 positives over 307 residues).

Nucleotide sequence of the region downstream of the promoter of parental strain, GP204. In order to identify the gene(s) endogenously expressed by the promoter directing expression of the CRR insert of GP1223, genomic walks downstream of the promoter region on parental strain GP204 were performed using either specific primer CF15, CF26, or CF 35, respectively, in combination with adaptor primer CF6 (FIG. 6B). The resulting PCR walks 6-15 (1637-bp), 6-26 (1409-bp) and 6-35 (1152-bp) were cloned into pCR2.1 and their nucleotide sequence was determined. The nucleotide sequence of all three PCR walks was identical from the regions in which the PCR products overlapped and downstream of the putative promoter were sequences that encoded two complete ORFs (designated ORF A and ORF B). ORF A and ORF B were predicted to encode polypeptides of 145 and 156 amino acid residues respectively. Homology searches of ORF A or ORF B against either the Non-redundant GenBank database ( ) or the Unfinished Microbial genomes database ( ) using the BLAST program were performed. ORF A bore homology over the C-terminal half of the predicted protein to the regulatory protein, SlyA, found in Escherichia coli, Salmonella typhimurium and other Enterobacteriaceae. The alignment of ORF A with SlyA(EC) contained 31 % identities (23 identities over 74 residues) and 49% positives (37 positives over 74 residues). SlyA is a member of the MarR family of transcriptional regulators and a BLOCKS search ( ) revealed ORF A to be a member of the MarR family as well. The search of the unfinished microbial genomes database revealed only one highly homologous predicted protein in the TIGR-1313 (sp12 contig) Streptococcus pneumoniae database that contained 85% residue identities (122 identities over 143 residues) and 90% positives (129 positives over 143 residues).

Similar searches with the predicted peptide sequence of ORF B revealed no known functional homologies or patterns. However, the search of the unfinished microbial genomes database also revealed only one highly homologous predicted protein in the TIGR-1313 Streptococcus pneumoniae database that contained 90% residue identities (140 identities over 155 residues) and 93% positives (145 positives over 155 residues). This ORF in the S. pneumoniae database was located in the same contig as the ORF A homologue described above (sp12) and the ORF B homologue was located immediately downstream of ORF A homologue revealing conservation in structural arrangement between the two subspecies as well.

While the invention has been described and illustrated herein by references to various specific material, procedures and examples, it is understood that the invention is not restricted to the particular material, combinations of material, and procedures selected for that purpose. Numerous variations of such details can be implied and will be appreciated by those skilled in the art.

1. A vector for expression of foreign genes in gram positive bacteria, comprising orfA and orfB.

2. A vector according to claim 1, wherein said gram positive bacteria are selected from the group consisting of Streptococcus gordonii, Streptococcus pyogenes, Streptococcus mutans, Streptococcus epidermidis, Streptococcus pneumoniae, Lactococcus lactis, Lactobacillis helveticus, Lactobacillis paracasei, Enterococcus faecalis, Staphylococcus aureus, Group B streptococci, Group G streptococci, Peptostreptococcus magnus, Streptococcus dysgalactiae, Streptococcus suis, Streptococcus sobrinus, Listeria monocytogenes, Actinomyces viscosis, Actinomyces naeslundii, Streptococcus zooepidemicus, Streptococcus equisimilis, Streptococcus sobrinus, Bacillus licheniformis, Streptococcus sanguis, and Streptococcus salivarius.

3. A vector according to claim 2, wherein said gram positive bacteria are Streptococcus gordonii.

4. A vector according to claim 1, wherein said vector further comprises an insertion site between orfA and orfB.

5. A vector according to claim 4, wherein said insertion site is selected from the group consisting of Ndel, BamHl, BgIII, Clal, EcoRI, EcoRV, HindIII, Hpal, Kpnl, Pvull, Pstl, Sacl, Sall, Scal, Spel, Sphl, Stul, Xbal, and Xhol.

6. A vector according to claim 5, wherein said insertion site is Ndel.

7. A vector according to claim 4, wherein said vector further comprises a DNA molecule encoding a peptide, polypeptide, or protein foreign to the gram positive bacteria, said DNA molecule being located between orfA and orfB, and following the insertion site.

8. A vector according to claim 7, wherein said peptide, polypeptide, or protein is the M6 protein.

9. A vector according to claim 1, further comprising a selectable marker.

10. A vector according to claim 9, wherein the selectable marker is an antibiotic resistance gene.

11. A vector according to claim 10, wherein said antibiotic resistance gene confers resistance to kanamycin, erythromycin, spectromycin, and/or tetracycline.

12. A vector according to claim 11, wherein the antibiotic resistance gene is selected from the group consisting of aphIII, ermC, ermAM, aadA, tetM, and tetO.

13. A vector for expression of foreign genes in gram positive bacteria, comprising nucleotides encoding amino acids 94 and 95 of the lacG gene.

14. A vector according to claim 13, wherein said gram positive bacteria are selected from the group consisting of Streptococcus gordonii, Streptococcus pyogenes, Streptococcus mutans, Streptococcus epidermidis, Streptococcus pneumoniae, Lactococcus lactis, Lactobacillis helveticus, Lactobacillis paracasei, Enterococcus faecalis, Staphylococcus aureus, Group B streptococci, Group G streptococci, Peptostreptococcus magnus, Streptococcus dysgalactiae, Streptococcus suis, Streptococcus sobrinus, Listeria monocytogenes, Actinomyces viscosis, Actinomyces naeslundii, Streptococcus zooepidemicus, Streptococcus equisimilis, Streptococcus sobrinus, Bacillus licheniformis, Streptococcus sanguis, and Streptococcus salivarius.

15. A vector according to claim 14, wherein said gram positive bacteria are Streptococcus gordonii.

16. A vector according to claim 13, wherein said vector further comprises an insertion site between the nucleotides encoding amino acids 94 and 95 of the lacG gene.

17. A vector according to claim 16, wherein said insertion site is selected from the group consisting of Ndel, BamHl, BgIII, Clal, EcoRI, EcoRV, HindIII, Hpal, Kpnl, Pvull, Pstl, Sacl, Sall, Scal, Spel, Sphl, Stul, Xbal, and Xhol.

18. A vector according to claim 17, wherein said insertion site is Ndel.

19. A vector according to claim 16, wherein said vector further comprises a DNA molecule encoding a peptide, polypeptide, or protein foreign to the gram positive bacteria, said DNA molecule being located between the nucleotides encoding amino acids 94 and 95 of the lacG gene, and following the insertion site.

20. A vector according to claim 19, wherein said peptide, polypeptide, or protein is the M6 protein.

21. A vector according to claim 13, further comprising a selectable marker.

22. A vector according to claim 21, wherein the selectable marker is an antibiotic resistance gene.

23. A vector according to claim 22, wherein said antibiotic resistance gene confers resistance to kanamycin, erythromycin, spectromycin, and/or tetracycline.

24. A vector according to claim 23, wherein the antibiotic resistance gene is selected from the group consisting of aphIII, ermC, ermAM, aadA, tetM, and tetO.
